(19) United States
(12) Patent Application Publication
(10) Pub. No.: US 2010/0311097 A1
(43) Pub. Date: Dec. 9, 2010

(54) STABLE ISOTOPE LABELED POLYPEPTIDE STANDARDS FOR PROTEIN QUANTITATION

(75) Inventor: Norman L. Anderson, Washington, DC (US)

Correspondence Address: Perkins Coie LLP, 607 Fourteenth Street, NW, Washington, DC, 20005, US

(73) Assignee: ANDERSON FORSCHUNG GROUP LLC, Washington, DC (US)

(21) Appl. No.: 12/698,827

(22) Filed: Feb. 2, 2010

Related U.S. Application Data
(63) Continuation of application No. 11/147,397, filed on Jun. 8, 2005, now abandoned.
(60) Provisional application No. 60/578,274, filed on Jun. 9, 2004.
(60) Provisional application No. 60/602,908, filed on Aug. 18, 2004.

(51) Int. Cl. C12Q 1/37; C07K 7/08; C07K 14/00

(52) U.S. Cl. 435/23; 530/326; 530/325; 530/324

(57) ABSTRACT

This invention relates to proteins having an amino acid sequence containing several amino acid subsequences found in nature and wherein at least two different subsequences act as monitor sequences, said subsequences being part of at least one natural protein which is a target protein, wherein the end of each of said two different subsequences have a cleavage site that will be cleaved by the same site-specific proteolytic treatment to release said subsequences.

This application takes priority from U.S. Provisional Patent Application 60/578,274 filed Jun. 9, 2004, and U.S. Provisional Patent Application 60/602,908 filed Aug. 19, 2004.

### FIELD AND BACKGROUND OF THE INVENTION

This invention relates to quantitative assays for evaluation of proteins in complex samples such as human plasma, and specifically to the generation and use of labeled peptides as Stable Isotope Standards (SIS). It would be useful to be able to produce large numbers of different SIS peptides more cheaply than can be accomplished by chemical synthesis, to purify them more efficiently than can be accomplished by individual HPLC purification, and to quantitate them by some means more efficiently than amino acid analysis of each peptide individually. Here I describe a strategy for making sets of SIS standards by protein expression. The invention can be used both for analysis of samples from a single individual source or, for purposes of evaluating the level of a particular protein in a population, can be used to analyze pooled samples from the target population.

There is a need for quantitative assays for proteins in various complex protein samples, e.g., in human plasma, serum and urine. Conventionally these assays have been implemented as immunoassays, making use of specific antibodies against target proteins as specificity and detection reagents. The current expansion of the diagnostic proteome suggests that the use of many protein measurements together as a panel provides superior diagnostic information compared to a single protein: here patterns of change can be associated with disease or treatment, instead of relying on single protein markers interpreted alone. This development presages the need to assay many more proteins than is currently feasible with existing immunoassays. New methods, particularly involving internal standardization with isotopically labeled peptides, allow mass spectrometry (MS) to provide large panels of such quantitative peptide and protein assays (as MS does in the measurement of low molecular weight drug metabolites currently). The efficient production, quantitative calibration and use of such standards remains an issue, however. The present invention addresses this problem by providing improvements in the manufacturing of multiple peptide standards, arranging such standards in fixed stoichiometries, and using them efficiently in assays of complex protein and peptide samples.

A general mass-spectrometry-based approach to protein quantitation involves digesting the proteins (e.g., with trypsin) into peptides that can be further fragmented (MS/MS) in a mass spectrometer to generate a sequence-based identification. The approach can be used with either electrospray (ESI) or MALDI ionization, and is typically applied after one or more dimensions of chromatographic fractionation to reduce the complexity of peptides introduced into the MS at any given instant. Optimized systems of multidimensional chromatography, ionization, mass spectrometry and data analysis (e.g., the multidimensional protein identification technology, or “MudPIT” approach of Yates, also referred to as shotgun proteomics) have been shown to be capable of detecting and identifying ˜1,500 yeast proteins in one analysis (Washburn, Wolters and Yates, Nat Biotechnol 19:242-7, 2001), while a single dimensional LC separation, combined with the extremely high resolution of a Fourier-transform ion cyclotron resonance (FTICR) MS identified more than 1,900 protein products of distinct open reading frames (i.e., predicted proteins) in a bacterium. In human urine, a sample much more like plasma than the microbial samples mentioned above, Patterson used a single LC separation ahead of ESI-MS/MS to detect 751 sequences derived from 124 different gene products. Recently, Adkins et al have used two chromatographic separations with MS to identify a total of 490 different proteins in human serum (Adkins, Varnum, Auberry, Moore, Angell, Smith, Springer and Pounds, Mol Cell Proteomics 1:947-55, 2002), and Anderson et al combined four datasets to generate a list of 1,175 non-redundant plasma components (Anderson, Polanski, Pieper, Gatlin, Tirumalai, Conrads, Veenstra, Adkins, Pounds, Fagan and Lobley, Mol Cell Proteomics 2004). Such methods should have the ability to deal with the numerous post-translational modifications characteristic of many proteins in plasma, as demonstrated by the ability to characterize the very complex post-translational modifications occurring in aging human lens (MacCoss, McDonald, Saraf, Sadygov, Clark, Tasto, Gould, Wolters, Washburn, Weiss, Clark and Yates, Proc Natl Acad Sci USA 99:7900-5, 2002). Since 1995 a single peptide has been used as a surrogate for the presence of a parent protein (from which the peptide was derived by proteolytic digestion) in a complex protein mixture, based on, e.g., MALDI-PSD (Griffin, MacCoss, Eng, Blevins, Aaronson and Yates, Rapid Commun Mass Spectrom 9:1546-51, 1995) or ion trap (Yates, Eng, McCormack and Schieltz, Anal Chem 67:1426-36, 1995) MS/MS spectra. Regnier et al have pursued an equivalent “signature peptide” quantitation approach (Chakraborty and Regnier, J Chromatogr A 949:173-84, 2002, Zhang, Sioma, Wang and Regnier, Anal Chem 73:5142-9, 2001), also the subject of a published patent application (Regnier, F. E., X. Zhang, et al. US 2002/0037532), in which protein samples are digested to peptides by an enzyme, differentially labeled with isotopically different versions of a protein reactive agent, purified by means of a selective enrichment column, and combined for MS analysis using MALDI or ESI-MS.

The protein discovery methods described above focus on identifying peptides and proteins in complex samples, but they generally offer poor quantitative precision and reproducibility when used without internal standards. The well-known idiosyncrasies of peptide ionization arise in large part because the presence of one peptide can affect the ionization and, thus, signal intensity of another. These have been major impediments to accurate quantitation by mass spectrometry. This problem can be overcome, however, through the use of stable isotope-labeled internal standards. At least four suitable isotopes (²H, ¹³C, ¹⁵N, ¹⁸O) are commercially available in suitable highly enriched (>98 atom %) forms. In principle, abundance data as accurate as that obtained in MS measurement of drug metabolites with internal standards (coefficients of variation <5%) should ultimately be obtainable. In the early 1980's ¹⁸O-labeled enkephalins were prepared and used to measure these peptides in tissues at ppb levels. In the 1990's GC/MS methods were developed to precisely quantitate stable isotope-labeled amino acids, and hence protein turnover, in human muscle and plasma proteins labeled in vivo. The extreme sensitivity and precision of these methods suggested that stable isotope approaches could be applied in quantitative proteomics investigations, given suitable protein or peptide labeling schemes.

Over the past several years, a variety of such labeling strategies have been developed. The most straightforward approach (incorporation of label to a high substitution level during biosynthesis), has been successfully applied to microorganisms (Lahm and Langen, Electrophoresis 21:2105-14, 2000) and mammalian cells in culture, but is unlikely to be usable directly in humans for cost and ethical reasons. A related approach (which is applicable to human proteins) is the now-conventional chemical synthesis of monitor peptides containing heavy isotopes at specific positions. Post-synthetic methods have also been developed for labeling of peptides to distinguish those derived from an “internal control” sample from those derived from an experimental sample, with a labeled/unlabeled pair subsequently being mixed and analyzed together by MS. These methods include Aebersold's isotope-coded affinity tag (ICAT) approach, (Goodlett, Keller, Watts, Newitt, Yi, Purvine, Eng, von Haller, Aebersold and Kolker, Rapid Commun Mass Spectrom 15:1214-21, 2001) as well as deuterated acrylamide and iodoacetamide for labeling peptide sulfhydrals, deuterated acetate to label primary amino groups, n-terminal-specific reagents, permethyl esterification of peptides carboxyl groups, and addition of twin ¹⁸O labels to the c-terminus of tryptic peptides during cleavage.

An early quantitative MS-based assay for a peptide was published in 1989 by Jardine et al (Lisek, Bailey, Benson, Yaksh and Jardine, Rapid Commun Mass Spectrom 3:43-6, 1989). The reference discloses use of a single stable isotope labeled peptide (substance P sequence. Prepared by chemical peptide synthesis) spiked into neuronal tissue, followed (after extraction from the tissue) by binding to an immobilized anti-substance-P-specific antibody, to enrich the neuropeptide substance P, and finally quantitation by MS. Substance P abundance was calculated from the ratio of natural peptide ion current to the internal labeled standard peptide of the same sequence: i.e., demonstrating all elements of the single analyte peptide standard/antibody enrichment process. Jardine et al used a 10-fold molar excess of the labeled version of substance P to act as both internal standard and carrier, and measured masses by fast-atom bombardment (FAB) selected-ion monitoring (SIM) MS. Crowther published a similar approach in 1994 (Crowther, Adusumalli, Mukherjee, Jordan, Abuaf, Corkum, Goldstein and Tolan, Anal Chem 66:2356-61, 1994) to detect peptide drugs in plasma using deuterated synthetic internal standards. Rose used synthetic stable isotope labeled insulin to standardize an MS method for quantitation of insulin (a small protein or large peptide), in which the spiked sample was separated by reverse phase chromatography to fractionate the sample. Gygi used stable-isotope-labeled synthetic peptides to quantitate the level of phosphorylated vs non-phosphorylated peptides in the digest of a protein isolated on a 1-D gel (Stemmann, Zou, Gerber, Gygi and Kirschner, Cell 107:715-26, 2001, Gerber, Rush, Stemman, Kirschner and Gygi, Proc Natl Acad Sci USA 100:6940-5, 2003) and has described a method for peptide quantitation (WO03016861) that uses the approach of Jardine with the addition of greater mass spectrometer resolution (selected reaction monitoring [SRM] in which the desired peptide is isolated by a first mass analyzer, the peptide is fragmented in flight, and a specific fragment is detected using a second mass analyzer). In each of these cases, the labeled peptide standards have been made by conventional solid-phase peptide synthesis.

The instant invention uses several of the cited methods of the prior art together with other technologies related to cell-free protein synthesis in an entirely novel combination. In the descriptions that follow, quantitation of proteins, peptides and other biomolecules is addressed in a general sense, and hence the invention disclosed is in no way limited to the analysis of plasma and other body fluids.

### SUMMARY OF THE INVENTION

The present invention provides methods for the production, purification, characterization and use of stable-isotope-labeled peptide sequences which can be used together or separately as internal standards in the mass spectrometric quantitation of peptides and proteins. Briefly, one or more monitor peptide sequences are selected to represent each protein to be measured (the “analytes”). In the case of trypsin cleavage of the analyte-containing sample, candidate monitor peptides will be tryptic peptides (i.e., generally ending in K or R). A set of selected monitor peptide sequences representing multiple protein analytes is then concatenated to yield an extended amino acid sequence (a “polySIS” sequence) that can be reverse-translated to yield a DNA sequence, which can be prepared by chemical DNA synthesis and incorporated into an expression vector. Appropriate polySIS-containing vectors can be introduced into any of a variety of cell-based (e.g., E coli) or cell-free (e.g., E. coli or rabbit reticulocyte) expression systems capable of linked transcription and translation, wherein the protein can be produced. Stable isotope labels can be incorporated into the polySIS protein product by providing as substrates to the expression system either a heavily isotope-substituted nutrient source (for a cell based system), or one or more heavily isotope-substituted amino acids (for an in vitro cell-free system). In either case isotopically-enriched ¹⁵N or ¹³C (preferably >99%) can be used as the input label to achieve a highly substituted product. The polySIS protein can be purified using specific tags incorporated into the expression vector sequence (e.g., poly-histidine at one or both ends or internally between SIS sequences) or based on physical properties such as solubility or size (i.e., on an SDS electrophoresis gel).

The intact polySIS protein can be quantitated once by amino acid analysis, yielding a molar concentration that applies to all the component SIS peptides subsequently liberated by proteolysis, thereby saving the cost and effort of individual amino acid analysis of each peptide separately. The polySIS protein can be added at known amounts to complex protein samples prior to proteolytic digestion, and digested with the sample proteins to produce a series of SIS peptides whose stoichiometry to one another is known, and whose absolute concentration is also known. Alternatively the polySIS can be pre-digested to yield a stoichiometric mixture of SIS peptides to be added to a sample before or after sample digestion. These SIS peptides are then used as standards for quantitation of sample protein derived peptides by mass spectrometry (e.g., as in the previously disclosed SISCAPA method disclosed in U.S. patent application Ser. No. 10/676,005 “High Sensitivity Quantitation of Peptides by Mass Spectrometry”).

### BRIEF DESCRIPTION OF THE DRAWINGS

FIG. 1 shows a schematic diagram of the process for designing and producing polySIS proteins, beginning with a set of protein targets (analytes to be measured by MS).

FIG. 2 shows examples of four monitor peptides.

FIG. 3 shows a series of additive terms defining an index used to prioritize tryptic peptides in silico.

FIG. 4 shows monitor peptide sequences chosen to represent 30 proteins associated with cardiovascular disease and some of their relevant properties.

FIG. 5 shows DNA sequence of the assembled polySIS synthetic gene, and the corresponding amino acid sequence translated in the correct frame.

FIG. 6 shows the complete amino acid sequence of the expressed polySIS protein CVD_{—}1, including n-terminal and c-terminal regions added by expression from the pIVEX2.4d vector.

FIG. 7 is a diagram showing the use of a polySIS protein.

### DETAILED DESCRIPTION OF THE INVENTION

A principle object of the current invention is to provide a convenient means for producing stable-isotope-labeled peptide standards useful in quantitative analysis of a mixture of peptides (typically a proteolytic digest of a complex protein sample such as human serum or plasma). The object is to produce such standards by a method that 1) is less expensive overall than conventional individual synthesis approaches, 2) allows more efficient purification (many SIS at once instead of one at a time), 3) provides an efficient means of assaying the quantity of the standard in absolute terms, and 4) ensures proper stoichiometry of a series of different SIS standards.

The terms “analyte”, and “ligand” may be any of a variety of different molecules, or components, pieces, fragments or sections of different molecules that one desires to measure or quantitate in a sample.

The term “monitor fragment” may mean any piece of an analyte up to and including the whole analyte which can be produced by a reproducible fragmentation process (or without a fragmentation if the monitor fragment is the whole analyte) and whose abundance or concentration can be used as a surrogate for the abundance or concentration of the analyte.

The term “monitor peptide” means a peptide chosen as a monitor fragment of a protein or peptide, and is typically a peptide of length 8-24 amino acids resulting from proteolytic treatment of the analyte (or target) protein.

The terms “proteolytic treatment” or “enzyme” may refer any of a large number of different enzymes, including trypsin, chymotrypsin, lys-C, V8 protease and the like, as well as chemicals, such as cyanogen bromide. In this context, a proteolytic treatment acts to cleave peptide bonds in a protein or peptide in a sequence-specific manner, generating a collection of shorter peptides (a digest).

The term “denaturant” includes a range of chaotropic and other chemical agents that act to disrupt or loosen the 3-D structure of proteins without breaking covalent bonds, thereby rendering them more susceptible to proteolytic treatment. Examples include urea, guanidine hydrochloride, ammonium thiocyanate, as well as solvents such as acetonitrile, methanol and the like.

The term “reverse-phase matrix” and “C18” are meant to include any of a variety of hydrophobic surface phases (such as C18 or C8 aliphatic hydrocarbons) presented on the surface of a solid support and in contact with aqueous solvent.

The terms “internal standard”, “isotope-labeled monitor fragment”, or “isotope-labeled monitor peptide” may be any altered version of the respective monitor fragment or monitor peptide that is 1) recognized as equivalent to the monitor fragment or monitor peptide in any separation process employed before MS detection and 2) differs from it in a manner that can be distinguished by a mass spectrometer, either through direct measurement of molecular mass or through mass measurement of fragments (e.g., through MS/MS analysis), or by another equivalent means.

By a “SIS” or “stable isotope standard” I mean a peptide internal standard having a unique sequence derived from a protein of interest and including a label of some kind (e.g., a stable isotope) that allows its use as an internal standard for quantitation (see U.S. patent application Ser. No. 10/676,005 “High Sensitivity Quantitation of Peptides by Mass Spectrometry”).

By “polySIS” I mean a polypeptide or protein composed of multiple SIS peptide sequences, and which may or may not include stable isotope labels.

The term “multiple reaction monitoring”, abbreviated MRM, means a mass spectrometric assay based on two stages of mass selection. In MRM, the first mass analyzer within the MS (MS1, also called quadrupole 1 or Q1) is set to pass the parent molecule (the monitor peptide), rejecting components of other mass-to-charge ratios (m/z). The monitor peptide is then fragmented in a collision chamber and passed to a second mass analyzer (MS2, also called quadrupole 3 or Q3) set to pass a known specific fragment of the monitor peptide. This two-stage selection of parent and fragment ions (selected reaction monitoring: SRM, plural MRM) affords great specificity, with the result that the detected signal usually traces a peak in the chromatogram at the expected retention time corresponding to the selected analyte. Integrating this peak gives a measure of the quantity of the analyte.

The term “cell-free” expression system means a combination of molecules capable of producing protein from an input DNA sequence. Examples include, but are not limited to, cell-free extracts of bacteria (like E coli) or eukaryotic cells (like rabbit reticulocytes) containing transcription and translation systems, together with appropriate accessory activities required to make mRNA and protein.

### Embodiments

In each of the following embodiments, it is to be assumed that the preferred method of use can include other elements of the SISCAPA system described in US2003/031126.

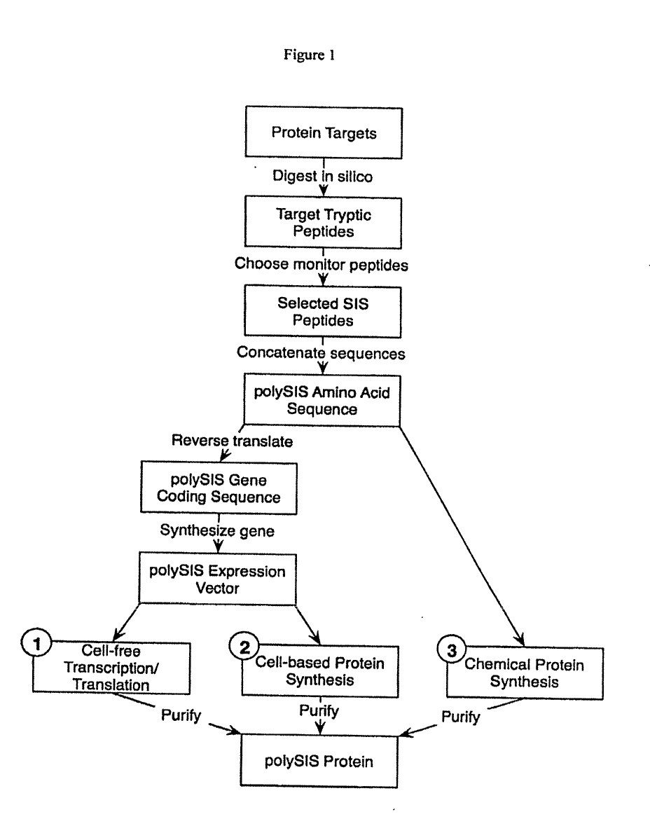

1) In a first embodiment, a polySIS protein is prepared according to the steps shown in FIG. 1 (track 1). First a set of protein targets is selected whose amounts or concentrations are to be measured in one or more samples. These targets are “digested” in silico using an algorithm appropriate for the desired protease (e.g., for trypsin cut at K and R, except where followed by P) to yield a set of target tryptic peptides. From these candidate peptides, monitor peptides may be selected using information including the predicted physical properties of these peptides and available experimental data (e.g., which “fly” best in a mass spectrometer), selecting those optimal properties for detection, enrichment, etc. Multiple peptides can be selected from a single target protein in order to provide multiple independent measurements of the target, thus improving measurement statistics.

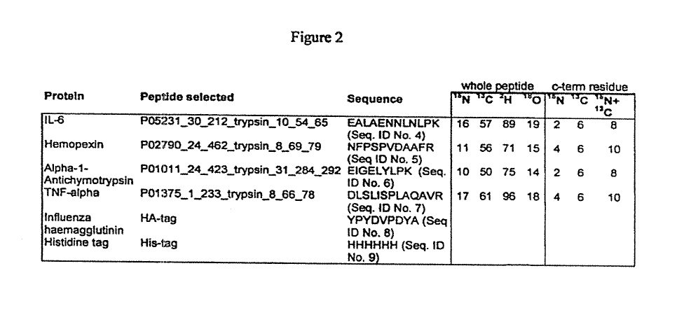

The monitor peptide sequences selected for use as stable isotope labeled internal standards (SIS), each including the cleavage site-defining K or R residue recognized by trypsin, are concatenated together in silico to yield a single polypeptide sequence. The number of peptides combined in this way can range from 2 to 100 or more, depending on the number of monitor peptides required to provide adequate measurements of the set of protein targets selected. In this embodiment, each monitor peptide sequence is included once in the concatenated polypeptide (although multiple copies of one monitor peptide can be used to achieve different, but integral, stoichiometries). The order of the monitor peptide sequence in the concatenated polypeptide is not of great significance, provided that the final proteolytic digestion is complete, as desired. Some adjustment of peptide order may be required if concatenation brings together sequences that inhibit complete cleavage at every intended cleavage site. Optionally, additional peptide sequences may be added to one or both ends of the concatenated monitor peptide sequence to provide “handles” for use in specific affinity purification of the concatenated protein product. For example, influenza hemagglutinin (HA) tag sequences can be added at one or both ends of the polySIS product to assist in purification of the polySIS protein. The tag sequences are separated from the n- and c-terminal monitor peptides by protease (e.g., trypsin) cleavage sites (“separator sequences”; e.g., the added K in FIG. 2) so that the tags are separated from the monitor peptides upon digestion. Multiple different purification tags may be used (e.g., HA and polyhistidine tags in FIG. 2 case 2). Different monitor peptide sequences may be included in different copy numbers in order to achieve different (integral) stoichiometries upon digestion (FIG. 2., case 4)

The complete polySIS sequence (comprising the monitor peptides, optional purification tags, and any required separator sequences) is reverse-translated into a DNA sequence using the appropriate genetic code, with codon usage optimized for translation in a suitable production organism such as E coli or a cell-free system based on E coli or rabbit reticulocytes, to yield a polySIS gene coding sequence.

Next, this DNA sequence is synthesized to produce a double-stranded polySIS DNA sequence (“polySIS gene”) using commercially available services and expertise (e.g., Blue Heron Biotechnology, GeneScript Corp., or SeqWright Inc.). In this process, the polySIS gene may be introduced into a temporary vector to facilitate generation of more DNA, or introduced directly into an expression vector appropriate for expression in a coupled in vitro transcription/translation system. A 1 kb DNA sequence (approximately 330 amino acids) is easily produced by current commercial technology, and can accommodate 30 SIS peptides of 11 amino acids. Codon usage is preferably optimized to suit the source of the translation system (e.g., E coli). In this embodiment, the polySIS expression vector (e.g., Roche Applied Science pIVEX2.4d vector) includes additional sequences required to initiate transcription (e.g., by a bacterial or phage DNA-dependent RNA polymerase), initiate translation on the resulting RNA (ribosome binding and translation initiation sites) and stop translation (a stop codon). This DNA construct can be made entirely by synthesis and ligation, without the need for cloning into a vector, or the extra sequences can be included in a vector optimized for in vitro transcription/translation.

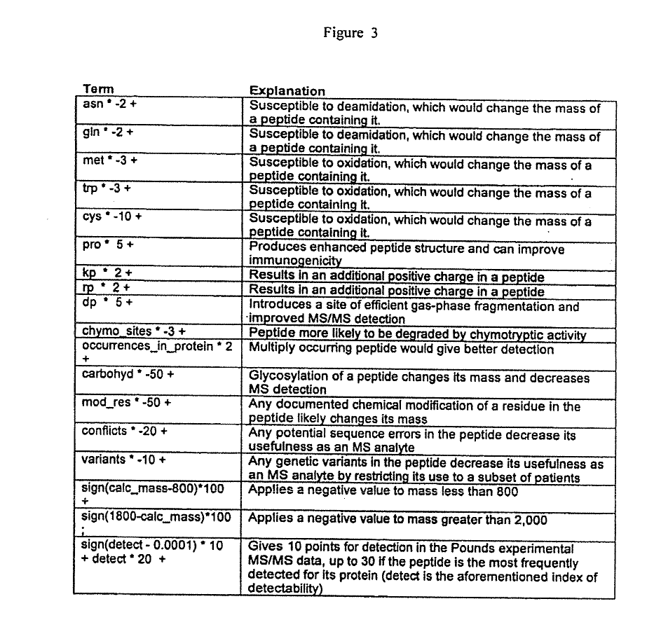

In either case, the polySIS molecule is introduced into a suitable linked in vitro transcription/translation system (e.g., the commercially available systems based on E coli or rabbit reticulocyte lysates) and polySIS protein product is generated. The translation system used preferably requires an exogenous source of amino acids, and in this embodiment at least one amino acid is provided that contains a stable isotope at high enrichment. The different SIS sequences comprising the polySIS product contain varying amino acids, and thus the mass increments in the various peptides resulting from use of a collection of labeled amino acids can be quite variable. A useful simplification results, however, if labeled K and R are used exclusively, since each tryptic SIS peptide contains only one such residue (either K or R) per peptide (except for rare cases in which a KP or RP occurs within the peptide). Using this K/R labeling approach, each SIS peptide is 6 amu heavier than the natural version if K and R fully substituted with 13C is used, or 2 and 4 amu respectively if K and R fully substituted with ¹⁵N is used, or 8 and 10 if K and R fully substituted with both isotopes are used (FIG. 3). A difference of at least 6 amu is preferred so that the SIS and natural peptides are far enough apart to avoid any overlap of SIS with the normal isotopic distribution of the natural unlabeled form. The polySIS protein product formed in the linked transcription/translation system is purified for use as an internal standard as described in the first embodiment.

Standard techniques, including affinity capture by chelated nickel adsorbents (in the case of histidine tags) or immobilized anti-HA antibodies (in the case of HA tags). The polySIS protein is recovered in a state of high purity (preferably greater than 95%). Alternatively a physical separation such as SDS gel electrophoresis can be used, and the polySIS protein band excised. An aliquot of purified polySIS protein is hydrolyzed in HCl to liberate amino acids, and these are quantitated by amino acid analysis to establish the absolute amount of polySIS protein present. Alternatively the polySIS protein can be assayed by other means such as quantitation of a substituent such as biotin introduced at fixed stoichiometry during synthesis. Using this quantitative information, solutions or dried aliquots of polySIS containing accurately known amounts of material are prepared as standards.

A known amount of polySIS (i.e., a known volume of standardized solution) is then added to a measured volume of a sample of proteins in which the target proteins are to be quantitated (in this case a sample of human blood plasma). This combined sample, including spiked polySIS standard, is then proteolytically digested by exposure to trypsin using any of a variety of well-known protocols. In one such protocol, plasma is denatured by addition of 9 volumes of 6 M guanidinium HCl/50 mM Tris-HCl/10 mM dithiothreitol and incubation for 2 hr at 60° C.; addition of 1 volume of 200 mM iodoacetamide followed by incubation for 30 min at 25° C.; addition of 1 volume of 200 mM dithiothreitol followed by incubation for 30 min at 25° C.; dilution to <1 M guanidinium HCl by addition of 50 mM NaHCO₃, addition of sequencing grade modified trypsin (e.g., from Promega, Madison, Wis.) at a 1:50 ratio (trypsin:plasma protein) and incubation overnight at 37° C. Digestion is allowed to proceed until substantially complete, liberating the monitor peptides from both target proteins and polySIS protein essentially to completion. Alternatively a mixture of SIS resulting from prior digestion of polySIS protein can be added to the sample before or after sample digestion. This sample digest now contains versions of monitor peptides containing natural isotopes (from peptides derived from the original sample) and stable isotopes (in the SIS peptides derived from the polySIS protein). In this embodiment, each SIS sequence is present only once in the polySIS product, and thus each is present at the same stoichiometry (i.e., the same number of moles per volume) as the initial polySIS standard added to the sample before digestion (after correction for any dilution or concentration occurring during or after the digestion protocol). Each sample-derived natural monitor peptide can then be quantitated by measuring its concentration relative to the stable isotope version (which has a known absolute concentration calculable from the amount spiked into the sample or sample digest), and this then allows calculation of the concentration of the associated target protein in the initial sample (as described in published U.S. patent application 20040072251, High sensitivity quantitation of peptides by mass spectrometry, Anderson, Norman. L). The relative concentrations of natural and stable isotope labeled monitor peptides are preferably measured by mass spectrometry as the relative ion currents recorded for the two peptides or their fragmentation products. The two versions perform essentially identically in any chromatographic or affinity based separation or enrichment process (provided N, C or O are used as labels), and thus co-elute, facilitating direct comparison of ion currents. In this embodiment, one polySIS protein replaces an entire collection of separate SIS peptides described in earlier disclosures, and eliminates the requirement to synthesize, purify, and standardize concentrations of the separate SIS peptide reagents. Quantitative MS measurements can be made using a variety of ionization sources (e.g., electrospray ionization [ESI] and matrix-assisted laser desorption ionization [MALDI]) and mass analyzers (e.g., time-of-flight [TOF], triple quadrupole [TQMS], Fourier transform ion cyclotron resonance [FTICR], and ion trap).

2) In a second embodiment, the process of the first embodiment is altered so as to use a vector suitable for expression in a selected cell-based expression system (FIG. 1, track 2). This vector, containing the polySIS coding sequence in the correct frame and orientation is introduced into the cells of such an expression system (e.g., E coli cells), which transcribe the polySIS gene into mRNA and translate this mRNA into a polySIS protein with high efficiency. In the case of E coli, additional sequences can be designed into the polySIS product to target it to the periplasmic space or to render it insoluble so as to form inclusion bodies. The E coli growth medium provided during the growth and product synthesis phase includes nutrients wherein at least one of the elements N, C, O or H is present in the form of an enriched (98% isotopic purity) stable isotope (¹⁵N, ¹³C, ¹⁸O or ²H respectively), thus ensuring that the polySIS product contains a high proportion of one or more stable isotopes. Under such conditions, SIS sequences such as the Hx and AAT peptides (FIG. 2 case 1 and FIG. 3) have masses greater than the natural versions by respectively 11 and 10 amu (if ¹⁵N is used) or 56 and 50 amu (if ¹³C is used). Once sufficient protein is produced, the cells are harvested, disrupted using conventional techniques, the protein contents recovered and the polySIS protein purified, making use of purification tags optionally included in the sequence.

3) In a third embodiment (FIG. 1, track 3)), the polySIS amino acid sequence of concatenated monitor peptides is synthesized using well-known methods of chemical peptide synthesis. These are typically carried out on a solid phase resin (Merrifield, Methods Enzymol 289:3-13, 1997), and can include steps to ligate together multiple synthetic peptides to produce larger, 30-100 kD proteins (Dawson, Muir, Clark-Lewis and Kent, Science 266:776-9, 1994, Dawson and Kent, Annu Rev Biochem 69:923-60, 2000). As in the first embodiment, the preferred case makes use of stable isotope labeled K and R, since each tryptic SIS peptide contains only one such residue (either K or R) per peptide. Incorporation of labeled K or R is achieved through use of the corresponding labeled K or R synthons commercially available for solid phase peptide synthesis. Alternatively any amino acid containing stable isotope labels can be used.

4) In a fourth embodiment, multiple polySIS products are made in order to facilitate standardized measurement of proteins having widely different abundances in the sample. Thus a first polySIS product can include monitor peptide sequences derived from proteins having expected concentrations around 1 mg/ml in human plasma (e.g., hemopexin and alpha-1-antichymotrypsin: (FIG. 2, case 3) while a second polySIS product is made containing monitor peptide sequences from low abundance (e.g., 10-1000 pg/ml) proteins such as IL-6 and TNF-alpha. Since the mass spectrometer detection systems used to measure the relative abundances of natural and SIS peptides have limited dynamic range (typically 100 to 1000), it is preferred to add an amount of each SIS peptide close to the expected amount of the equivalent natural monitor peptide. Thus the second polySIS described would optimally be added at a level approximately 1,000,000-fold less than the first polySIS above. In cases where the numbers of SIS peptides required in quantitative studies exceed the number that can conveniently be prepared as one polySIS protein, due to limitations on protein product size in many cell-free and solid phase chemical synthesis approaches, it is natural and efficient to group the desired SIS peptides into classes according to the expected concentration of the proteins from which they arise in the sample. If a set of monitor peptides were selected within a decade of concentration range (i.e., all members within a factor of 10 in expected concentration), then 6 polySIS products would be required to span a total dynamic range of 1,000,000 between the most and least abundant target protein. Six such products would accommodate a total of 200 or more SIS sequences if each were limited to a synthesized gene length of 1 kb.

5) In a fifth embodiment, unequal stoichiometries between individual SIS peptides are achieved by the incorporation of more than one copy of some SIS sequences in a polySIS product in which two copies of one SIS are concatenated with one copy of another SIS). In this case, exact ratios between the amounts of different SIS peptides are be achieved by virtue of the necessarily integral numbers of copies present in the gene and the protein. Thus a polySIS product with 1 copy of a SIS sequence denoted A, 2 copies of B, 4 copies of C and 10 copies of D can provide peptide standards at concentrations that match the amounts of monitor peptides derived from proteins expected to be present at relative concentrations of 1:2:4:10 in the original sample. Many approaches will be apparent to those skilled in the art for inserting multiple copies of specific SIS sequences into a polySIS gene.

6) In a sixth embodiment, two or more monitor peptide sequences are selected from the digest products of a single target analyte protein, and SIS sequences for each of these are incorporated into the polySIS product, but at different ratios. Thus SIS sequences A, B and C from a given target protein may be incorporated into the polySIS at multiplicities of 1 copy (A), 4 copies (B) and 16 copies (C). These three SIS peptides then provide an effective standard curve for measuring target protein concentration and establishing linearity over a range of at least 16-fold and generally more. The natural monitor peptides corresponding to SIS A, B and C will be present in equal amounts (in the typical case where one molecule of each is derived by digestion from one molecule of the target protein), and thus will be detected at consistent ratios versus the SIS standards: e.g., the ratios of natural monitor:SIS standard for A, B and C sequences will be x:1, x:4 and x:16. Use of multiple monitor peptides provides improved measurement precision through better statistics, and better accuracy through use of a multipoint calibration curve.

7) In a seventh embodiment, calibrants for quantitative mass spectrometry are provided. Here two polySIS sequences are created each comprising the same series of peptides (which can be monitor peptides but can be other sequences as well). One polySIS sequence (here called X) may be comprised of a single copy of each component monitor sequence (i.e., sequences A,B,C,D present at 1,1,1,1 copies), and is produced without an incorporated stable isotope label. The other polySIS sequence may be comprised of the same monitor sequences but present in different copy numbers, e.g., A,B,C,D present in 1,2,4,8 copies respectively, and produced in an expression system so as to incorporate a stable isotope label. When equal numbers of molecules of the first and second polySIS are combined and digested to release SIS sequences, the peptide sequences A,B,C,D will each be present in unlabeled (from the first polySIS) and labeled (from the second polySIS) forms. These forms will be present in precise quantitative ratios of 1:1 (A), 1:2 (B), 1:4 (C) and 1:8 (D). These accurately defined ratios provide a precise means for calibrating the linearity of response of the mass spectrometer.

8) In an eighth embodiment, DNA sequences for SIS peptides are inserted into “cassettes” allowing them to be joined into expressible polySIS genes by standard molecular biology techniques. These include the techniques of recombinational cloning as well as PCR-based methods. This approach allows a series of SIS peptide sequences to be assembled into polySIS genes in different ways (i.e., different orders or at different multiplicities) by DNA fragment manipulation rather than by repeated synthesis of the entire polySIS gene.

9) In a ninth embodiment, an easily assayed substituent is incorporated into the polySIS during synthesis and used for later quantitation of the polySIS protein. An example is the incorporation of a single biotin group into a specific lysine of the polySIS through use of the Roche “RTS AviTag Biotinylation Reagents for Enzymatic Monobiotinylation of Proteins”. This site is added to the polySIS protein through use of the appropriate pIVEX vector. The presence of the biotin group at 1 mole per mole of protein then allows absolute quantitation of the polySIS standard protein through use of a standard assay for the biotin tag (e.g., a competition assay using immobilized streptavidin as capture agent and a biotinylated acid phosphatase as the competing ligand able to generate a colorimetric signal). In addition, the biotin tag can be used for purification of the bulk polySIS protein by binding to a streptavidin column. The polySIS can be released from such a column by selective elution or by cleavage at a peptide sequence linking the SIS sequences to the biotinylated site using a specific protease (e.g., Factor Xa) with a specificity different from the protease used to liberate SIS (e.g., trypsin).

10) In a tenth embodiment, entire domains of target proteins are combined into the polySIS instead of short peptides. In this approach, each domain contains at least one and preferably several peptides (e.g., tryptic peptides), and thus offers multiple opportunities to quantitate the target. More importantly, by including entire domains likely to fold in a manner more similar to the fold of part of the intact whole target protein, the polySIS better replicates the environment within which the proteolysis will occur for the native target protein—i.e., the cleavage of the peptides in the polySIS is likely to better parallel the efficiency in the target.

11) In an eleventh embodiment, polySIS digestion products (SIS peptides), either labeled or unlabeled, are used as test materials for the optimization of MS/MS detection of the peptides. Since the relative abundances of various fragments produced in MS/MS is difficult to predict, and since one wants to maximize the production and detection sensitivity of a specific parent/fragment mass pair (particularly in triple quadrupole selected reaction monitoring as a quantitation technique), the availability of test samples of each selected target peptide provides a valuable test material for tuning MS parameters. By digesting the polySIS and infusing the resulting mix of the selected SIS peptides in a continuous infusion experiment, one can select one SIS (target) sequence at a time and systematically vary MS parameters (e.g., collision energy, mass selection windows, etc) to maximize detection of any of its fragments. One can also systematically select the best fragment for each SIS peptide in terms of detection sensitivity, signal-to-noise, and limit of quantitation. This optimization can improve the lower limit of quantitation (LLOQ) of an MS assay by a factor of 10 or more.

### Example

A series of 177 proteins and protein forms that are demonstrated or potential plasma markers of some aspect of cardiovascular disease was assembled (Anderson, J Physiology 563.1:23-60, 2005). Protein sequence information for the candidate markers was obtained using Swissprot accession numbers in two stages. First, when the protein was already listed in the non-redundant list of human plasma proteins described previously (Anderson, Polanski, Pieper, Gatlin, Tirumalai, Conrads, Veenstra, Adkins, Pounds, Fagan and Lobley, Mol Cell Proteomics 2004), the relevant accession in that non-redundant set was used. If the protein was not in this list, it was located, where possible, by query of the Swissprot web database using protein names, and added to the non-redundant list. In some cases the name used in the literature was not sufficiently specific to allow selection of a single gene product, and the candidate was not taken forward. Sequence and Swissprot annotation data was obtained in text format from the Swissprot server (http://au.expasy.org/sprot/sprot-retrieve-list.html) and placed in a relational database implemented using the postgreSQL open-source database software running on an Apple Macintosh Powerbook G4 computer. Database functions were written in the PL/pgSQL language to parse the Swissprot information into fields containing the sequence, annotation related to the beginning and end of the mature protein (the CHAIN, SIGNAL, PEPTIDE and PROPEPTIDE descriptors), as well as the presence of sites where the sequence is modified in ways relevant to MS of peptides (the MOD RES, CONFLICT, VARIANT, CARBOHYD descriptors). A separate sequence table was constructed using a PL/pgSQL function to extract that part of each sequence defined by a Swissprot CHAIN, PEPTIDE or PROPEPTIDE annotation and store it as a possible mature protein product. The “mature” products thus obtained were labeled as the Swissprot accession followed by the starting and ending amino acid positions separated by underscore characters (e.g., P08519_{—}20_{—}4548 for the CHAIN of Apolipoprotein(a)), and each was tagged with the name of that segment (e.g., haptoglobin alpha and beta chains, derived from a single translation product) in the Swissprot annotation (important where a single protein product is cleaved to yield multiple sequences with different names and functions).

Additional PL/pgSQL functions were used to “digest” each mature protein “in silico” to yield a list of its predicted tryptic peptides (29,155 total entries), which were stored in a separate table. Of these, 21,609 peptides occurred in only a single protein within the set of plasma proteins, and, because monitor peptides used for protein quantitation should uniquely represent a single protein analyte, only these peptides were carried forward for further analysis. The number of occurrences of each peptide in its parent protein was tabulated (in some cases more than one), in order to provide a conversion factor between moles of protein and moles of each peptide derived from it. The tryptic digestion algorithm cleaved a protein at each Arg or Lys residue, except those followed by Pro. The peptides generated were labeled by extending the mature product name with the “enzyme” used and the beginning and ending amino acid positions of the peptide within the mature sequence (e.g., P08519_{—}20_{—}4548_trypsin_{—}110_{—}2071_{—}2080).

Computation of peptide parameters. Using a combination of PL/pgSQL functions and SQL steps, a series of parameters was calculated for each of the 21,609 peptides and stored in the database. Amino acid composition was obtained by counting the number of occurrences of each amino acid in a peptide, as was the number of occurrences of important dipeptides such as KP and RP (the only occurrences of K and R inside our predicted peptide sequences) and DP, a site within which peptide fragmentation is predicted to be especially efficient, yielding intense MS/MS signals. Peptide mass was computed in the same way as for the whole proteins, i.e., from the amino acid composition and the amino acid masses. Hoop-Woods hydrophilicity was computed by summing the standard coefficients for each residue weighted by the number of the corresponding amino acid residues (Hopp and Woods, Proc Natl Acad Sci USA 78:3824-8, 1981). A predicted retention time in reversed-phase (C18) chromatography was computed using the algorithm of Krokhin (Krokhin, Craig, Spicer, Ens, Standing, Beavis and Wilkins, Mol Cell Proteomics 3:908-19, 2004). Likely chymotryptic cleavages sites were counted. Several additional peptide attributes proved useful in the final selection process. An index of the likelihood of experimental detection was derived from a data set reported by Adkins (Adkins, Varnum, Auberry, Moore, Angell, Smith, Springer and Pounds, Mol Cell Proteomics 1:947-55, 2002): peptides detected in that MS/MS analysis of serum were given values equal to the number of separate “hits” for the peptide in the data set divided by the number of hits for the most frequently detected peptide from the same protein. Thus the index ranged from 1.0 for the most frequently detected peptide in a protein down to 0.1 or less for minor but still detected peptides. Predicted tryptic peptides that were not detected experimentally in the Pounds data set were given index values of 0.0. Normal plasma protein concentration values obtained from the literature were converted to a uniform scale (pg/ml). For multi-subunit proteins (e.g., fibrinogen composed of alpha, beta and gamma subunits) a factor was generated that reflected the fraction of the normal concentration attributable to that subunit. Finally a figure was derived for the molar concentration of these proteins, expressed as fmol/ml. The molar concentration of each peptide derived from such proteins is equal to the protein molar concentration times the number of occurrences of the peptide within the protein sequence. Since in some cases particular peptides occur many times (e.g., GTYSTTVTGR (Seq. ID No. 2) occurs 31 times in apolipoprotein (a) —P08519_{—}20_{—}4548), this correction is critical to obtaining accurate quantitative values. It also suggests that peptides of high multiplicity should yield improved detectability compared to singly represented peptides, all other factors being equal.

An overall index was generated by combining the various quantitative features described above according to a formula in which various favorable numerical criteria (e.g., content of proline) were multiplied by positive coefficients, while unfavorable criteria were multiplied by negative coefficients (FIG. 3). Peptides derived from each target protein were ranked by the overall index resulting from this formula and finally selected manually through consideration of several additional criteria in addition to the rank. Peptides that are preceded by a dipeptide of (K or R) were avoided where possible to avoid the likelihood of incomplete trypsin cleavage at KK, RR, KR and RK and thus lack of stoichiometric release of the monitor peptide from the target protein. The proteins were ranked according to plasma concentration on a molar basis, beginning with albumin and decreasing towards the low abundance cytokines. The objective was to select monitor peptides for a series of protein targets, starting at the high abundance end of the distribution and extending downwards.

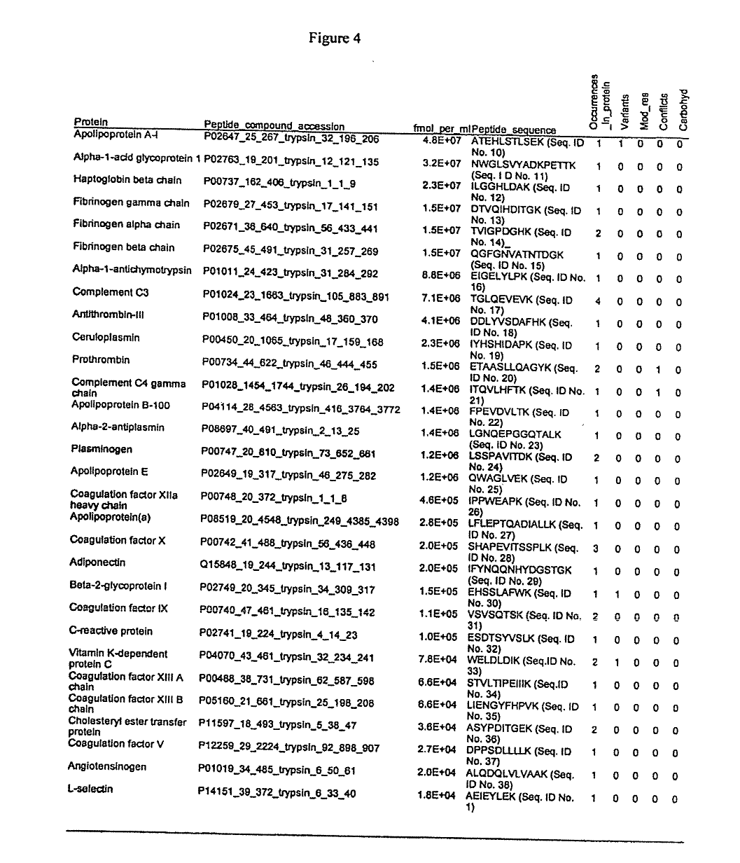

A practical polySIS gene length of 1,000 bases (selected due to commercial availability through synthesis) can code for 333 amino acids, which, given the average size of peptides selected here for MS/MS (8-14 amino acids), allows polySIS products comprising 28 to 30 SIS peptides. Two different sets of monitor peptides were selected for each of a set of 30 protein marker candidates (FIG. 4) selected from among the candidate markers of cardiovascular disease: one set of peptides ending in c-terminal Arg and one ending in Lys (the two amino acids at which trypsin cleaves). The mass increment due to full ¹³C and ¹⁵N labeling of the c-terminal amino acid is 8 amu for Lys and 10 amu for Arg, both sufficient to ensure adequate separation from the natural peptide isotopic distribution to give good quantitation by MS. In this example, Lys peptides were selected for further study for inclusion in polySIS protein CVD_{—}1. In general it is possible to select good peptides having few recorded post-translational modifications (mod_res), genetic variants, sequence conflicts or glycosylation sites (carbohyd), the existence of which would alter the MS properties of the monitor peptide and disturb the equivalence of the labeled (polySIS) and unlabeled (sample-derived) versions in at least some samples.

It was noted that 5 of the final monitor peptides selected for the polySIS sequence occurred unmodified in the mouse cognate protein sequence, and thus could be useful in quantitative standardization of plasma measurements in that species. The other human sequences, which do not appear to occur in the mouse proteome, could be useful as negative quantitative controls (for which there should be no corresponding peptides in mouse plasma).

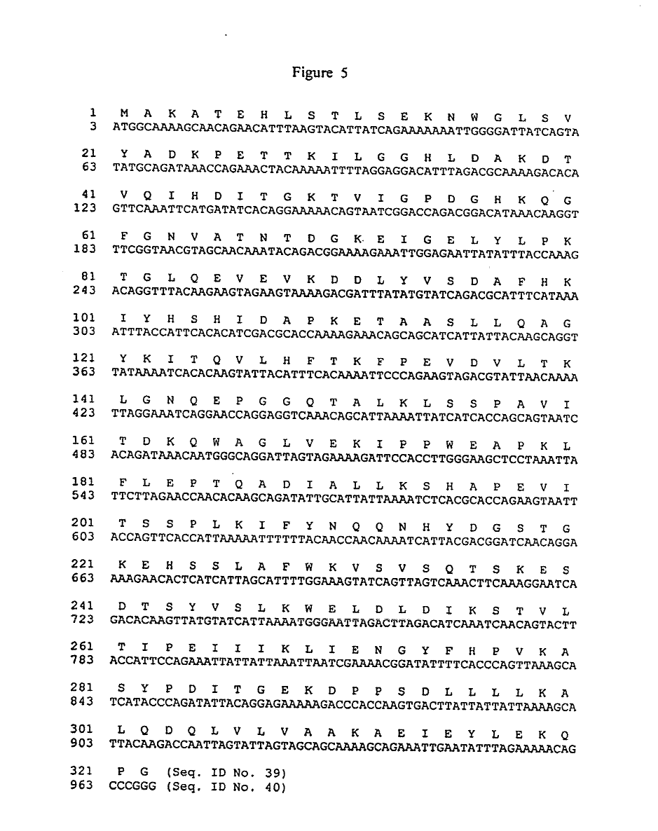

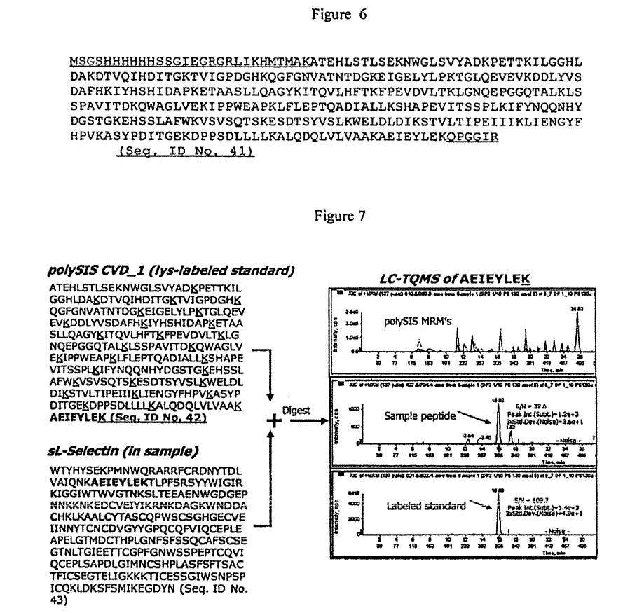

The selected Lys-ending monitor peptide sequences were concatenated into a linear sequence, in this case ordered from high to lower expected target abundance. The first peptide was preceded by an added Lys in order to release it from n-terminal vector-provided sequence. The CVD_{—}1 amino acid sequence was backtranslated into a DNA sequence, optimizing codon usage for the E. coli-based cell-free system, avoiding NcoI and SmaI sites in the coding region in order to permit their use for cloning later, and introducing short 3′ and 5′ extensions providing appropriate restriction enzyme recognition sites. A synthetic CVD_{—}1 gene (FIG. 5) was synthesized commercially (Blue Heron Technologies, Bothell, Wash.) and amplified by PCR using gene specific oligos with a 15 by overhang specific to the pIVEX2.4d vector (Roche Applied Science, Indianapolis, Ind.). The template was digested with DpnI and the remaining PCR product purified. The amplified gene was mixed with pIVEX2.4d that had been linearized with NcoI and SmaI, and ligated into the vector with Clontech's In-Fusion Cloning enzyme (BD Biosciences Clontech, Mountain View, Calif.). This vector provides an n-terminal His6 purification tag and Factor Xa protease site in the expressed protein (sequence in FIG. 6). The predicted CVD_{—}1 protein has a computed molecular mass of 38,525.76, a computed pI of 6.08, and should yield 35 tryptic peptides (5 arising from the c- and n-terminal extensions plus the 30 monitor peptides).

It will be clear from this example that a wide variety of known and novel vectors could be used as vehicles for expression of the polySIS sequence, in both cell-based and cell-free expression systems, and to amplify it, and that a plethora of cloning strategies could be used to insert the polySIS sequence into a vector. It is also possible to expand the polySIS by PCR without use of a cloning vector.

For convenience, it is advantageous to arrange that the mass increment added to each of the labeled SIS peptides in comparison with its natural version is the same for all peptides. This can be achieved by arranging that one amino acid is labeled, and that this amino acid occurs only once per peptide. Since trypsin cleaves at most Lys and Arg residues, these are the obvious choices for labeling. Use of a single labeled amino acid also allows production of the polySIS protein, and the SIS peptides it comprises, most economically, since the cost of each different labeled amino acid is substantial. In the case of lysine, a version in which all 6 carbons are replaced with ¹³C and both nitrogens with ¹⁵N (U-¹³C₆ U-¹⁵N₂: a total mass increment of 8 amu compared to the natural peptide) is available commercially at high (98-99%) substitution levels. As described above, a different set of monitor peptides could have been selected ending in Arg, for which an analogous commercial product is available with 10 amu mass increment.

The positioning of the label atoms at the extreme c-terminus of each peptide has the effect that all fragments that contain the c-terminus (i.e., the y-ions) will show the mass shift due to the label, whereas all the fragments that contain the n-terminus (and hence have lost one of more c-term residues: the b-series ions) will have the same masses as the corresponding fragments from the natural (sample-derived) target protein. These features (shifted y-ions, normal b-ions) provide a simplification in interpreting the fragmentation patterns of the SIS peptides. By selecting y-ions for use in relative quantitation of labeled (SIS) and sample-derived, unlabeled monitor peptides of the same sequence, the ions have identical properties except for a shift of 8 amu (for the Lys label used here). This mass increment appears as a +4 amu shift for +2 charge peptides (z=2), and +813 amu for +3 charged peptides (z=3).

An E. coli-based cell-free expression system (Roche Applied Science “RTS” coupled transcription/translation system) was used to produce the polySIS protein CVD_{—}1. Use of a cell-free system avoids the interconversion between labeled and unlabeled amino acids that occurs in cell-based systems. Recent advances in the output of cell-free systems have made it possible to prepare milligram quantities of protein by this route: quantities sufficient to provide polySIS for many analyses given that 1 mg of the 38.5 kD polySIS is 26 nmol of product, or 29,000,000,000 amol (where 100 amol is a quantifiable amount of peptide in MS/MS). The RTS cell-free approach (commercially available kit) was used, with a mixture of 19 unlabeled amino acids and labeled lysine (U-¹³C₆ U-¹⁵N₂ labeled: +8 amu).

Once all the reagents were mixed, the plasmid was added and the reaction proceeded for 18 hours at 30 C and shaking at 750 rpm in a RTS ProteoMaster (Roche Applied Science). The CVD_{—}1 polysis protein proved to be insoluble (despite having been constructed from relatively hydrophilic peptides) and was recovered as a major component of the pellet after centrifugation. Although the protein contains purification tags, no tag-based purification was used here.

When polySIS CVD_{—}1 was digested with trypsin, the peptides modified with o-methylisourea and analyzed by MALDI-MS, ten of the expected peptides were detected at the expected masses, accounting for a majority of the observed peaks in the appropriate mass range. When the polySIS digest was analyzed by reversed-phase liquid chromatography and tandem mass spectrometry (using an Applied Biosystems 4000 Q-TRAP linear ion trap instrument), all 30 expected SIS peaks were observed at the expected masses (typically as doubly-charged ions). Using MS/MS data acquired on the SIS peptides, multiple reaction monitoring (MRM) assays were devised for each, providing three parameters: parent ion mass (Q1, typically doubly-charged), a high-mass specific y-ion fragment (Q3, typically singly charged and thus having a higher m/z than the parent), and collision energy appropriate for fragmentation in the collision cell of the 4000 Q-TRAP instrument. MRM assay parameters for the sample-derived unlabeled monitor peptides were obtained by subtracting the mass increments due to the stable isotopes labels from the Q1 and Q3 mass parameters of the labeled SIS peptides. The MS/MS data was also analyzed to assess single cleavage failures by scanning for the presence of molecules containing any two adjacent SIS peptides. Only one such failure was detected at high abundance (the peptide ILGGHLDAKTVIGPDGHK (Seq. ID No. 3), containing SIS peptides 28 and 29 in the polySIS protein).

For use for internal standardization in peptide quantitation, an amount of the polySIS protein (the “spiked” standard) is added to a sample of plasma or serum. In this case, the polySIS protein was digested before addition of the resulting SIS peptide mixture to a digest of normal human plasma from which 6 major proteins had been previously subtracted using the Agilent MARS column. Quantitative mass spectrometry was used to measure the ratios between the ion currents of monitor peptides and same-sequence SIS standards using the 4000 Q-TRAP instrument in triple quadrupole mode. This ratio, when multiplied by the known concentration of the polySIS, provides the concentration of the monitor peptides, and thus of the target proteins in the sample at the time it was spiked.

Thus 1,300 amol of a tryptic digest of polySIS CVD_{—}1 protein (containing 1,300 amol of each of the 30 SIS peptides) was added to the peptides derived from digestion of 0.01 ul of normal human plasma (from which 6 major proteins had been previously subtracted). The resulting peptide mixture was injected onto a 75 micron diameter C18 reversed phased LC column (LC Packings, a division of Dionex, Sunnyvale Calif.), and eluted with a 40 minute gradient of 3-30% acetonitrile with 0.1% formic acid. A total of 137 MRM's were observed by time-slice multiplexing, and the peak areas of each obtained using Analyst software (Applied Biosystems). A set of 17 of the 30 SIS peptides were followed by specific MRM's, and of these 14 were detected at a signal-to-noise (S/N) ratio >10 (the usual criterion for quantitation in MS assays). The unlabeled, sample-derived same-sequence monitor peptides were detected at S/N >10 for 15 of the 17 SIS sequences, thus permitting calculation of the ratio of peak areas for SIS and monitor peptides for use in quantitation.

The peak areas for the L-selectin monitor peptide (AEIEYLEK (Seq ID No. 1)) and SIS standard were 17,620 and 79,930 respectively, yielding a ratio of 0.216. When multiplied by the 1,300 amol SIS loading, and considering that there is one copy of this peptide per molecule of intact L-selectin, this yields an L-selectin concentration of 280 amol per 0.01 ul, or 28 pmol/ml. Given a molecular weight for plasma L-selectin of ˜35,000, this gives a measured concentration of 980 ng/ml. This may be compared with the published normal value of 670 ng/ml obtained by immunoassay. Given that the L-selectin monitor peptide was detected with a signal-to-noise ratio of 22, and that the lower limit of quantitation (LLOQ) is generally defined as a S/N of 10, L-selectin could have been quantitated using this MS assay at a level of ˜450 ng/ml.

1-35. (canceled)

36. A labeled polypeptide chain comprising:
a plurality of amino acid sequences, wherein the ends of each of said sequences have a cleavage site that will be cleaved by the same site-specific proteolytic treatment,
wherein each said amino acid sequence consists of an 8-24 amino acid sequence that is identical to an amino acid sequence occurring within a polypeptide chain found within a biological sample,
wherein each of said amino acid sequences comprises at least one isotope label selected from the group consisting of 15N, 13C, 2H, and 180,
wherein each of said amino acid sequences occurs in a different naturally-occurring polypeptide chain in said biological sample and is unique to that protein among the proteins of the sample.

37. The labeled polypeptide according to claim 36 wherein said labeled polypeptide further comprises a detectable ligand that permits quantitation or purification of said protein.

38. The labeled polypeptide according to claim 37 wherein said detectable ligand is selected from the group consisting of biotin, a sulfhydryl group, a sugar moiety and a nucleic acid.

39. The labeled polypeptide according to claim 36 wherein said labeled polypeptide further comprises a peptide sequence that permits affinity purification of said polypeptide.

40. The labeled polypeptide according to claim 39 wherein said peptide sequence that permits affinity purification of said polypeptide is selected from the group consisting of an influenza hemagglutinin tag and polyhistidine.

41. The labeled polypeptide chain according to claim 36 wherein each of said amino acid sequences is substituted at greater than 98% isotopic purity with at least one isotope label selected from the group consisting of 15N, 13C, 2H, and 180.

42. The labeled polypeptide chain according to claim 36 wherein each of said cleavage sites is an enzymatic cleavage site.

43. The labeled polypeptide chain according to claim 42, wherein each of said cleavage sites is a site for cleavage by an enzyme selected from the group consisting of trypsin, Lys-C, Arg-C chymotrypsin, proteinase K, Asp N and Glu-C.

44. The labeled polypeptide chain according to claim 36, wherein each of said cleavage sites is a site for cleavage by cyanogen bromide, or BNPS-skatole.

45. The labeled polypeptide according to claim 36, wherein said labeled polypeptide comprises at least 28 amino acid sequences identical to an amino acid sequence occurring within a polypeptide chain found within a biological sample.

46. A composition comprising a labeled polypeptide according to claim 36 and a biological sample.

47. The composition according to claim 46 wherein said biological sample is plasma, serum, or urine.

48. The labeled polypeptide chain according to claim 36 wherein at least one amino acid in each of said amino acid sequences is substituted at greater than 98% isotopic purity with at least one isotope label selected from the group consisting of 15N, 13C, 2H, and 180.


SEQUENCE LISTING (43 sequences)

SEQ ID NO: 1 - protein, 8 aa, Artificial Sequence
  Description of Artificial Sequence Synthetic peptide
AEIEYLEK

SEQ ID NO: 2 - protein, 10 aa, Artificial Sequence
  Description of Artificial Sequence Synthetic peptide
GTYSTTVTGR

SEQ ID NO: 3 - protein, 18 aa, Artificial Sequence
  Description of Artificial Sequence Synthetic peptide
ILGGHLDAKTVIGPDGHK

SEQ ID NO: 4 - protein, 12 aa, Artificial Sequence
  Description of Artificial Sequence Synthetic peptide
EALAENNLNLPK

SEQ ID NO: 5 - protein, 11 aa, Artificial Sequence
  Description of Artificial Sequence Synthetic peptide
NFPSPVDAAFR

SEQ ID NO: 6 - protein, 9 aa, Artificial Sequence
  Description of Artificial Sequence Synthetic peptide
EIGELYLPK

SEQ ID NO: 7 - protein, 13 aa, Artificial Sequence
  Description of Artificial Sequence Synthetic peptide
DLSLISPLAQAVR

SEQ ID NO: 8 - protein, 9 aa, Artificial Sequence
  Description of Artificial Sequence Synthetic peptide
YPYDVPDYA

SEQ ID NO: 9 - protein, 6 aa, Artificial Sequence
  Description of Artificial Sequence Synthetic peptide
HHHHHH

SEQ ID NO: 10 - protein, 11 aa, Artificial Sequence
  Description of Artificial Sequence Synthetic peptide
ATEHLSTLSEK

SEQ ID NO: 11 - protein, 15 aa, Artificial Sequence
  Description of Artificial Sequence Synthetic peptide
NWGLSVYADKPETTK

SEQ ID NO: 12 - protein, 9 aa, Artificial Sequence
  Description of Artificial Sequence Synthetic peptide
ILGGHLDAK

SEQ ID NO: 13 - protein, 11 aa, Artificial Sequence
  Description of Artificial Sequence Synthetic peptide
DTVQIHDITGK

SEQ ID NO: 14 - protein, 9 aa, Artificial Sequence
  Description of Artificial Sequence Synthetic peptide
TVIGPDGHK

SEQ ID NO: 15 - protein, 13 aa, Artificial Sequence
  Description of Artificial Sequence Synthetic peptide
QGFGNVATNTDGK

SEQ ID NO: 16 - protein, 9 aa, Artificial Sequence
  Description of Artificial Sequence Synthetic peptide
EIGELYLPK

SEQ ID NO: 17 - protein, 9 aa, Artificial Sequence
  Description of Artificial Sequence Synthetic peptide
TGLQEVEVK

SEQ ID NO: 18 - protein, 11 aa, Artificial Sequence
  Description of Artificial Sequence Synthetic peptide
DDLYVSDAFHK

SEQ ID NO: 19 - protein, 10 aa, Artificial Sequence
  Description of Artificial Sequence Synthetic peptide
IYHSHIDAPK

SEQ ID NO: 20 - protein, 12 aa, Artificial Sequence
  Description of Artificial Sequence Synthetic peptide
ETAASLLQAGYK

SEQ ID NO: 21 - protein, 9 aa, Artificial Sequence
  Description of Artificial Sequence Synthetic peptide
ITQVLHFTK

SEQ ID NO: 22 - protein, 9 aa, Artificial Sequence
  Description of Artificial Sequence Synthetic peptide
FPEVDVLTK

SEQ ID NO: 23 - protein, 13 aa, Artificial Sequence
  Description of Artificial Sequence Synthetic peptide
LGNQEPGGQTALK

SEQ ID NO: 24 - protein, 10 aa, Artificial Sequence
  Description of Artificial Sequence Synthetic peptide
LSSPAVITDK

SEQ ID NO: 25 - protein, 8 aa, Artificial Sequence
  Description of Artificial Sequence Synthetic peptide
QWAGLVEK

SEQ ID NO: 26 - protein, 8 aa, Artificial Sequence
  Description of Artificial Sequence Synthetic peptide
IPPWEAPK

SEQ ID NO: 27 - protein, 14 aa, Artificial Sequence
  Description of Artificial Sequence Synthetic peptide
LFLEPTQADIALLK

SEQ ID NO: 28 - protein, 13 aa, Artificial Sequence
  Description of Artificial Sequence Synthetic peptide
SHAPEVITSSPLK

SEQ ID NO: 29 - protein, 15 aa, Artificial Sequence
  Description of Artificial Sequence Synthetic peptide
IFYNQQNHYDGSTGK

SEQ ID NO: 30 - protein, 9 aa, Artificial Sequence
  Description of Artificial Sequence Synthetic peptide
EHSSLAFWK

SEQ ID NO: 31 - protein, 8 aa, Artificial Sequence
  Description of Artificial Sequence Synthetic peptide
VSVSQTSK

SEQ ID NO: 32 - protein, 10 aa, Artificial Sequence
  Description of Artificial Sequence Synthetic peptide
ESDTSYVSLK

SEQ ID NO: 33 - protein, 8 aa, Artificial Sequence
  Description of Artificial Sequence Synthetic peptide
WELDLDIK

SEQ ID NO: 34 - protein, 12 aa, Artificial Sequence
  Description of Artificial Sequence Synthetic peptide
STVLTIPEIIIK

SEQ ID NO: 35 - protein, 11 aa, Artificial Sequence
  Description of Artificial Sequence Synthetic peptide
LIENGYFHPVK

SEQ ID NO: 36 - protein, 10 aa, Artificial Sequence
  Description of Artificial Sequence Synthetic peptide
ASYPDITGEK

SEQ ID NO: 37 - protein, 10 aa, Artificial Sequence
  Description of Artificial Sequence Synthetic peptide
DPPSDLLLLK

SEQ ID NO: 38 - protein, 12 aa, Artificial Sequence
  Description of Artificial Sequence Synthetic peptide
ALQDQLVLVAAK

SEQ ID NO: 39 - protein, 322 aa, Artificial Sequence
  Description of Artificial Sequence Synthetic polySIS protein
MAKATEHLSTLSEKNWGLSVYADKPETTKILGGHLDAKDTVQIHDITGKTVIGPDGHKQGFGNVATNTDGKEIGELYLPKTGLQEVEVKDDLYVSDAFHKIYHSHIDAPKETAASLLQAGYKITQVLHFTKFPEVDVLTKLGNQEPGGQTALKLSSPAVITDKQWAGLVEKIPPWEAPKLFLEPTQADIALLKSHAPEVITSSPLKIFYNQQNHYDGSTGKEHSSLAFWKVSVSQTSKESDTSYVSLKWELDLDIKSTVLTIPEIIIKLIENGYFHPVKASYPDITGEKDPPSDLLLLKALQDQLVLVAAKAEIEYLEKQPG

SEQ ID NO: 40 - DNA, 966 bp, Artificial Sequence
  Description of Artificial Sequence Synthetic polySIS gene
atggcaaaagcaacagaacatttaagtacattatcagaaaaaaattggggattatcagtatatgcagataaaccagaaactacaaaaattttaggaggacatttagacgcaaaagacacagttcaaattcatgatatcacaggaaaaacagtaatcggaccagacggacataaacaaggtttcggtaacgtagcaacaaatacagacggaaaagaaattggagaattatatttaccaaagacaggtttacaagaagtagaagtaaaagacgatttatatgtatcagacgcatttcataaaatttaccattcacacatcgacgcaccaaaagaaacagcagcatcattattacaagcaggttataaaatcacacaagtattacatttcacaaaattcccagaagtagacgtattaacaaaattaggaaatcaggaaccaggaggtcaaacagcattaaaattatcatcaccagcagtaatcacagataaacaatgggcaggattagtagaaaagattccaccttgggaagctcctaaattattcttagaaccaacacaagcagatattgcattattaaaatctcacgcaccagaagtaattaccagttcaccattaaaaattttttacaaccaacaaaatcattacgacggatcaacaggaaaagaacactcatcattagcattttggaaagtatcagttagtcaaacttcaaaggaatcagacacaagttatgtatcattaaaatgggaattagacttagacatcaaatcaacagtacttaccattccagaaattattattaaattaatcgaaaacggatattttcacccagttaaagcatcatacccagatattacaggagaaaaagacccaccaagtgacttattattattaaaagcattacaagaccaattagtattagtagcagcaaaagcagaaattgaatatttagaaaaacagcccggg

SEQ ID NO: 41 - protein, 350 aa, Artificial Sequence
  Description of Artificial Sequence Synthetic polySIS protein sequence
MSGSHHHHHHSSGIEGRGRLIKHMTMAKATEHLSTLSEKNWGLSVYADKPETTKILGGHLDAKDTVQIHDITGKTVIGPDGHKQGFGNVATNTDGKEIGELYLPKTGLQEVEVKDDLYVSDAFHKIYHSHIDAPKETAASLLQAGYKITQVLHFTKFPEVDVLTKLGNQEPGGQTALKLSSPAVITDKQWAGLVEKIPPWEAPKLFLEPTQADIALLKSHAPEVITSSPLKIFYNQQNHYDGSTGKEHSSLAFWKVSVSQTSKESDTSYVSLKWELDLDIKSTVLTIPEIIIKLIENGYFHPVKASYPDITGEKDPPSDLLLLKALQDQLVLVAAKAEIEYLEKQPGGIR

SEQ ID NO: 42 - protein, 316 aa, Artificial Sequence
  Description of Artificial Sequence Synthetic protein sequence
ATEHLSTLSEKNWGLSVYADKPETTKILGGHLDAKDTVQIHDITGKTVIGPDGHKQGFGNVATNTDGKEIGELYLPKTGLQEVEVKDDLYVSDAFHKIYHSHIDAPKETAASLLQAGYKITQVLHFTKFPEVDVLTKLGNQEPGGQTALKLSSPAVITDKQWAGLVEKIPPWEAPKLFLEPTQADIALLKSHAPEVITSSPLKIFYNQQNHYDGSTGKEHSSLAFWKVSVSQTSKESDTSYVSLKWELDLDIKSTVLTIPEIIIKLIENGYFHPVKASYPDITGEKDPPSDLLLLKALQDQLVLVAAKAEIEYLEK

SEQ ID NO: 43 - protein, 294 aa, Homo sapiens
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN